(19) United States
(12) Patent Application Publication
(10) Pub. No.: US 2026/0199075 A1
(43) Pub. Date: Jul. 16, 2026

(54) PREPARATION METHOD AND PREPARATION DEVICE FOR MICRO-HOOK OF SUSPENSION BELT WITH FIXING FUNCTION

(71) Applicants: CONDINER MEDICAL TECHNOLOGY CO., LTD., Changzhou, Jiangsu (CN); PEKING UNION MEDICAL COLLEGE HOSPITAL, BEIJING (CN)

(72) Inventors: Lan ZHU, Changzhou (CN); Yifeng ZHA, Changzhou (CN); Yang CAO, Changzhou (CN); Xiaoju CHEN, Changzhou (CN); Jun CHEN, Changzhou (CN)

(21) Appl. No.: 19/447,047

(22) Filed: Mar. 4, 2026

(30) Foreign Application Priority Data
Jan. 13, 2025 (CN) 2025100479156

(51) Int. Cl. A61F 2/00; B05C 19/00; D06C 13/08

(52) U.S. Cl. CPC A61F 2/0045; B05C 19/001; D06C 13/08; A61F 2220/0016; A61F 2240/001; D10B 2509/00

(57) ABSTRACT

A preparation method and a preparation device for a micro-hook of a suspension belt with a fixing function are disclosed. The preparation method for the micro-hook of the suspension belt with the fixing function includes coil breaking, surface melt-blowing and cooling; and the preparation device includes a coil breaking device, a melt-blowing device, a cooling device, a mesh unreeling device and a mesh reeling device.

### CROSS-REFERENCE TO RELATED APPLICATION

The present application claims the benefit and priority of the Chinese Patent Application No. CN2025100479156, filed on Jan. 13, 2025, which is herein incorporated by reference in its as part of the application.

### TECHNICAL FIELD

The present disclosure relates to the field of medical suspension belt manufacturing, and more particularly relates to a preparation method and a preparation device for a micro-hook of a suspension belt with a fixing function.

### BACKGROUND ART

At present, the most commonly used surgical treatment way for urinary incontinence is mid-urethral suspension and correction, which is one of the standard treatment methods for surgical treatment of female stress urinary incontinence. A strip-shaped meshed suspension belt is implanted in a tissue space between a lower part of a urethra and an anterior vaginal wall under the urethra by utilizing a guide handle, which may correct and reduce urethral swing. The implanted meshed suspension belt may also play an elastic suspension supporting role through certain viscosity and tissue reaction with tissue.

Existing suspension belts are mainly woven from materials such as polypropylene, and are prone to displacement and deformation after being implanted due to smooth surfaces thereof, thereby affecting a treatment effect. Arranging a micro-hook on a surface of one side of a mesh of the suspension belt is an effective means to improve a gripping force between the suspension belt and the tissue. At present, the suspension belts with a fixing function are mainly made of meshes with the micro-hooks, and such meshes are also applied in self-fixing hernia repair patches, for example, “repair patch woven fabric with barb, hernia repair mesh made therefrom, and method for manufacturing repair patch woven fabric” with the publication number CN104024508B, “hot-melt forming device for mushroom-head micro-hook of self-fixing medical repair patch” with the publication number CN215020025U, and the like. Most of the micro-hooks of these meshes form barb structures by melting and breaking coils on the woven meshes through high-temperature melting and ironing. However, the process stability of the high-temperature melting and ironing is poor, which is mainly reflected in relatively large fluctuations of melting and ironing temperatures. At low temperatures, as shown in (a) of FIG. 1, a phenomenon of incomplete melting is severe, thereby resulting in a reduction in the number of formed micro-hooks; while at high temperatures, as shown in (b) of FIG. 1, the coils are seriously drawn, the shapes and sizes of the micro-hooks are inconsistent, and mushroom heads at head ends of the micro-hooks have smooth surfaces, thereby resulting in relatively small frictional forces of the formed micro-hooks. All these situations will lead to relatively small overall gripping forces and relatively poor self-fixing performance of the meshes. Therefore, the above high-temperature melting and ironing process has a very high requirement for temperature control, and the quality of the formed micro-hooks varies greatly.

### SUMMARY OF THE DISCLOSURE

### 1. Technical Problem to be Solved by the Present Disclosure

An object of the present disclosure is to provide a preparation method and a preparation device for a micro-hook of a suspension belt with a fixing function, so as to overcome the above deficiencies of an existing preparation process for the micro-hook of the suspension belt. By adopting the technical solution of the present disclosure, coil breaking and melt-blowing processes are organically combined, and irregular fluff is formed on surfaces of short burrs formed by coil breaking and a mesh, thereby increasing a frictional force of the mesh, improving a gripping force of the suspension belt made of the mesh in a body, reducing the risk of the suspension belt sliding on tissue, reducing the suture fixation of the suspension belt during an operation, and saving the operation time; in addition, due to the existence of the melt-blown fluff, the requirements for shapes and sizes of barbs on the short burrs are reduced, and the efficiency and process flexibility of a coil breaking working procedure can be improved, thereby being conducive to improving the overall production efficiency of the mesh and the suspension belt.

### 2. Technical Solution

In order to achieve the above object, the technical solution provided by the present disclosure is as follows:
a preparation method for a micro-hook of a suspension belt with a fixing function of the present disclosure, includes the steps of:
S1. coil breaking: one surface of a mesh being provided with coils, and breaking the coils of the mesh from middle parts by utilizing a coil breaking device to form two short burrs after each of the coils is broken;
S2. surface melt-blowing: performing melt-blowing on a surface of the mesh with the short burrs by utilizing a melt-blowing device to form irregular fluff, and the fluff adhering to surfaces of the short burrs and the mesh; and
S3. cooling: cooling the mesh after being subjected to melt-blowing in step S2 by utilizing a cooling device or in a natural cooling manner to solidify the fluff.

Further, the coil breaking device is a laser cutting machine, a high-temperature melting and ironing machine, a cutting machine or a grinding machine.

Further, a melt-blowing material in step S2 is the same as a material of the mesh.

Further, the coil breaking device in step S1 melts and breaks the coils through a high temperature, and melts ends of the short burrs to form barbs.

Further, the mesh sequentially passes through the coil breaking device, the melt-blowing device and the cooling device, and the mesh moves continuously or intermittently among the coil breaking device, the melt-blowing device and the cooling device to sequentially complete coil breaking, surface melt-blowing and cooling working procedures.

Further, the mesh is woven by a weaving machine, the mesh woven by the weaving machine directly enters the coil breaking device, the melt-blowing device and the cooling device, and the processed mesh is collected by a reeling device.

A preparation device for a micro-hook of a suspension belt with a fixing function of the present disclosure, includes a coil breaking device, a melt-blowing device, a cooling device, a mesh unreeling device and a mesh reeling device, wherein the coil breaking device, the melt-blowing device and the cooling device are sequentially arranged between the mesh unreeling device and the mesh reeling device, a roll of mesh is mounted on the mesh unreeling device, and one end of the mesh is sequentially wound around the coil breaking device, the melt-blowing device and the cooling device and then connected to the mesh reeling device; one surface of the mesh is provided with coils, and the mesh moves under the action of the mesh unreeling device and the mesh reeling device; the coil breaking device is used to break the coils of the mesh from middle parts to form two short burrs after each of the coils is broken; the melt-blowing device is provided with a melt-blowing die head controlled to move along X-axis and Y-axis directions, the melt-blowing die head is used to perform melt-blowing on a surface of the mesh with the short burrs to form irregular fluff, and the fluff adheres to surfaces of the short burrs and the mesh; and the cooling device is used to cool the mesh after being subjected to melt-blowing to solidify the fluff.

Further, in the melt-blowing device, the melt-blowing die head is mounted on an XY-axis moving mechanism, a bracket is arranged below the melt-blowing die head, a transmission mechanism is arranged on the bracket, and a height adjustment mechanism used to adjust a height of the transmission mechanism is further arranged at a lower part of the bracket; and during melt-blowing, the mesh has a moving speed of 3-10 mm/s on the transmission mechanism, and the melt-blowing die head has a reciprocating moving speed of 3-10 mm/s in the X-axis direction, a reciprocating moving speed of 0.5-5 mm/s in the Y-axis direction, and a reciprocating moving amplitude of 3-10 mm in the Y-axis direction.

Further, a plurality of melt channels are uniformly distributed on the melt-blowing die head along the Y-axis direction, the melt channels are connected to melt material barrels, the melt-blowing die head is further provided with hot air channels positioned in two sides of the melt channels, and the hot air channels are connected to hot wind delivery devices.

Further, the coil breaking device is a laser cutting machine, including a laser cutting head, a supporting plate, a guide roller and a Y-axis moving mechanism, the mesh is tensioned on the supporting plate arranged along the Y-axis through the guide roller, a contact end of the supporting plate with the mesh is of a sharp-angle structure, so that the mesh positioned at a sharp-angle position of the supporting plate is in a “<” shape, the laser cutting head is mounted on the Y-axis moving mechanism, and the laser cutting head is driven by the Y-axis moving mechanism to move along the sharp-angle position of the supporting plate to cut off the coils on the mesh; and during laser cutting of the coils, the mesh has a moving speed of 3-10 mm/s on the supporting plate, an input power of the laser cutting head is controlled at 10-30 W, a Y-axis reciprocating moving speed is controlled at 10-30 mm/s, and a Y-axis cutting stroke of the laser cutting head is 250-450 mm.

### 3. Beneficial Effects

Compared with the existing prior art, the technical solution provided by the present disclosure has the following significant effects:
(1) for the preparation method and the preparation device for the micro-hook of the suspension belt with the fixing function of the present disclosure, a technological process includes coil breaking, surface melt-blowing and cooling, and by organically combining the coil breaking and melt-blowing processes, the irregular fluff is formed on the surfaces of the short burrs formed by coil breaking and the mesh, thereby increasing the frictional force of the mesh, improving the gripping force of the suspension belt made of the mesh in the body, reducing the risk of the suspension belt sliding on the tissue, reducing the suture fixation of the suspension belt during the operation, and saving the operation time; in addition, due to the existence of the melt-blown fluff, the requirements for the shapes and sizes of the barbs on the short burrs are reduced, and the efficiency and process flexibility of the coil breaking working procedure can be improved, thereby being conducive to improving the overall production efficiency of the mesh and the suspension belt;
(2) for the preparation method for the micro-hook of the suspension belt with the fixing function of the present disclosure, the coil breaking device may be the laser cutting machine, the high-temperature melting and ironing machine, the cutting machine or the grinding machine, which may not only melt and break the coils through the high temperature, but also break the coils by adopting mechanical cutting or grinding, with strong flexibility in equipment selection, thereby being conducive to improving the stability and efficiency of coil breaking and reducing production costs;
(3) for the preparation method for the micro-hook of the suspension belt with the fixing function of the present disclosure, the melt-blowing material is the same as the material of the mesh, which can better combine the melt-blown fluff with the mesh; and when the mesh is made of an absorbable material, and the fluff is also made of the absorbable material, which may be degraded and absorbed in the body, thereby reducing a foreign body sensation of the mesh in the body;
(4) for the preparation method for the micro-hook of the suspension belt with the fixing function of the present disclosure, the coil breaking device melts and breaks the coils through the high temperature, and melts the ends of the short burrs to form the barbs, and the barbs also have the melt-blown fluff, which may increase the sizes and surface frictional forces of the barbs, and further improve the gripping force between the suspension belt and the tissue;
(5) for the preparation method for the micro-hook of the suspension belt with the fixing function of the present disclosure, the mesh sequentially passes through the coil breaking device, the melt-blowing device and the cooling device, and the mesh moves continuously or intermittently among the coil breaking device, the melt-blowing device and the cooling device to sequentially complete coil breaking, surface melt-blowing and cooling working procedures, so that the production of the mesh is more continuous, and the production efficiency is improved; further, the mesh is woven by the weaving machine, the mesh woven by the weaving machine directly enters the coil breaking device, the melt-blowing device and the cooling device, and the processed mesh is collected by the reeling device, so that the intermediate reeling and unreeling working procedures are reduced, thereby further improving the production efficiency and reducing mesh loss;
(6) for the preparation device for the micro-hook of the suspension belt with the fixing function of the present disclosure, the melt-blowing die head is mounted on the XY-axis moving mechanism, the bracket is arranged below the melt-blowing die head, the transmission mechanism is arranged on the bracket, and the height adjustment mechanism used to adjust the height of the transmission mechanism is further arranged at the lower part of the bracket, so that a melt-blowing effect can be ensured by adjusting a melt-blowing distance, and the melt-blowing uniformity can be improved by cooperating with the X-axis and Y-axis reciprocating movement of the melt-blowing die head;
(7) for the preparation device for the micro-hook of the suspension belt with the fixing function of the present disclosure, the plurality of melt channels are uniformly distributed on the melt-blowing die head along the Y-axis direction, the melt channels are connected to the melt material barrels, the melt-blowing die head is further provided with the hot air channels positioned in the two sides of the melt channels, and the hot air channels are connected to the hot wind delivery devices; a melt material is sucked by adopting a negative-pressure suction force generated by high-pressure hot air passing through the melt-blowing die head, and the high-pressure hot air purges the melt material to enable the melt material to be diverged and sprayed outwards, thereby being conducive to improving the melt-blowing uniformity and the irregularity of the formed fluff; meanwhile, the high-pressure hot air can maintain a temperature of the melt-blowing die head, thereby ensuring the fluidity of the melt material and preventing the blockage of the melt channels; and
(8) for the preparation device for the micro-hook of the suspension belt with the fixing function of the present disclosure, the coil breaking device is the laser cutting machine, including the laser cutting head, the supporting plate, the guide roller and the Y-axis moving mechanism, the laser cutting machine is higher in cutting speed and more complete and stable in coil breaking, and a power of laser cutting may be reduced by cooperating with the melt-blowing process, thereby being conducive to reducing the risk of cutting a mesh substrate and ensuring the stability of the processing quality of the mesh.

### BRIEF DESCRIPTION OF THE DRAWINGS

FIG. 1 is a schematic structural diagram of a self-fixing micro-hook formed by an existing high-temperature melting and ironing process;

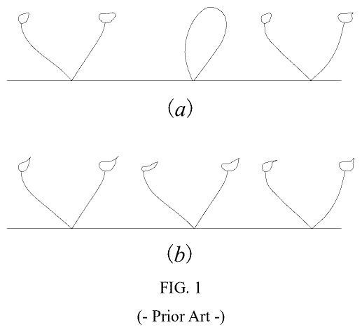

FIG. 2 is a technological process diagram of a preparation method for a micro-hook of a suspension belt with a fixing function of the present disclosure;

FIG. 3 is a schematic structural diagram of a self-fixing micro-hook formed by the preparation method for the micro-hook of the suspension belt with the fixing function of the present disclosure;

FIG. 4 is a schematic structural diagram of a preparation device for a micro-hook of a suspension belt with a fixing function of the present disclosure;

FIG. 5 is a schematic structural diagram of a melt-blowing device in the present disclosure;

FIG. 6 is a schematic diagram of a melt-blowing structure of a melt-blowing die head in the present disclosure;

FIG. 7 is a schematic diagram of a three-dimensional structure of the melt-blowing die head in the present disclosure; and

FIG. 8 is a structural principle diagram of breaking coils by adopting a laser cutting machine in the present disclosure.

### DETAILED DESCRIPTION OF THE INVENTION

In order to further understand the content of the present disclosure, the present disclosure is described in detail in conjunction with the accompanying drawings and examples.

### Examples

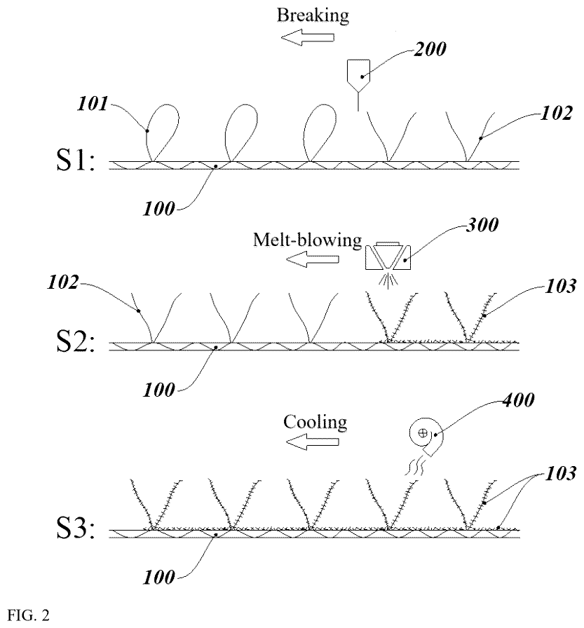

As shown in FIG. 2, a preparation method for a micro-hook of a suspension belt with a fixing function of this example, includes the steps of:
S1. coil breaking: one surface of a mesh 100 being provided with coils 101, the coils 101 being woven together during weaving of the mesh, and breaking the coils 101 of the mesh 100 from middle parts by utilizing a coil breaking device 200 to form two short burrs 102 after each of the coils 101 is broken; and one surface being provided with the short burrs 102 being a self-fixing surface of the mesh 100;
S2. surface melt-blowing: performing melt-blowing on a surface of the mesh 100 with the short burrs 102 by utilizing a melt-blowing device 300 to form irregular fluff 103, and the fluff 103 adhering to surfaces of the short burrs 102 and the mesh 100; and
S3. cooling: cooling the mesh 100 after being subjected to melt-blowing in step S2 by utilizing a cooling device 400 or in a natural cooling manner to solidify the fluff 103; wherein the hardened fluff 103 can increase the roughness of the surfaces of the mesh 100 and the short burrs 102 to form a plurality of tiny barb structures. The suspension belt with the fixing function is made of the mesh 100 prepared in the above steps S1 to S3.

Compared with an existing preparation method for the micro-hook of the suspension belt with the fixing function, for the preparation method for the micro-hook of the suspension belt with the fixing function of this example, the coil breaking and melt-blowing processes are organically combined, and the irregular fluff is formed on the surfaces of the short burrs formed by coil breaking and the mesh, thereby increasing a frictional force of the mesh, improving a gripping force of the prepared suspension belt in a body, reducing the risk of the suspension belt sliding on tissue, reducing the suture fixation of the suspension belt during an operation, and saving the operation time; in addition, due to the existence of the melt-blown fluff, the requirements for shapes and sizes of barbs on the short burrs are reduced, and the efficiency and process flexibility of a coil breaking working procedure can be improved, thereby being conducive to improving the overall production efficiency of the mesh and the suspension belt.

The coil breaking device 200 may adopt various manners, and only needs to achieve the breaking of the coils 101. In this example, the coil breaking device 200 may be a laser cutting machine, a high-temperature melting and ironing machine, a cutting machine or a grinding machine, and the like, wherein the laser cutting machine or the high-temperature melting and ironing machine adopts a high-temperature melting and breaking process, and the cutting machine or the grinding machine adopts a mechanical breaking process. The laser cutting machine achieves the breaking of the coils 101 by utilizing thermal energy of laser beams, the high-temperature melting and ironing machine irons and breaks the coils 101 by utilizing a high-temperature ironing plate or a high-temperature metal wire, the cutting machine cuts off the coils 101 by utilizing a blade rotating at a high-speed, and the grinding machine grinds and breaks the coils 101 by utilizing a grindstone or a grinding belt moving at a high-speed. Specifically, the coil breaking device 200 may be selected according to process requirements. The coils may be molten and broken by adopting the high temperature, and the coils may also be broken by adopting the mechanical cutting or grinding, with strong flexibility in equipment selection, thereby being conducive to improving the stability and efficiency of coil breaking and reducing production costs.

In this example, a melt-blowing material in step S2 is the same as a material of the mesh 100, which can better combine the melt-blown fluff 103 with the mesh 100. For the mesh 100 woven by blending absorbable monofilaments and non-absorbable monofilaments, the melt-blowing material is preferably the same absorbable material, such as polypropylene and other absorbable materials, so that the mesh 100 may be partially degraded and absorbed in the body, thereby reducing a foreign body sensation of the mesh of the suspension belt in the body.

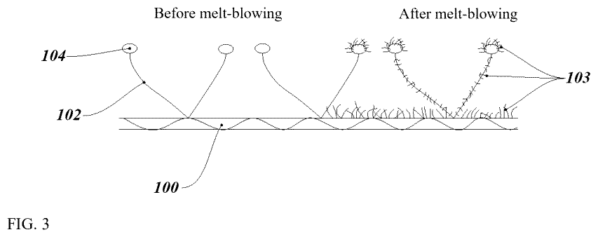

As a preferred embodiment, the coil breaking device 200 in step S1 described above melts and breaks the coils 101 through a high temperature, and melts ends of the short burrs 102 to form barbs 104. Specifically, the laser cutting machine or the high-temperature melting and ironing machine may be adopted, and a high-temperature effect may melt and shrink the ends of the short burrs 102 to form the barbs 104 with increased sizes at the ends of the short burrs 102 while breaking the coils 101 (as shown in FIG. 3). During melt-blowing, the irregular fluff 103 may also adhere to the barbs 104, and the fluff 103 may further increase the sizes and surface frictional forces of the barbs 104, thereby further improving a gripping force between the mesh of the suspension belt and the tissue.

In this example, the mesh 100 sequentially passes through the coil breaking device 200, the melt-blowing device 300 and the cooling device 400, and the mesh 100 moves continuously or intermittently among the coil breaking device 200, the melt-blowing device 300 and the cooling device 400 to sequentially complete coil breaking, surface melt-blowing and cooling working procedures, so that the production of the mesh of the suspension belt is more continuous, and the production efficiency is improved. The mesh 100 with the coils 101 may be an existing roll of mesh purchased from a supplier, and may also be directly woven by a weaving machine (such as a warp knitting machine). In a feasible embodiment, the mesh 100 is woven by the weaving machine, the mesh 100 woven by the weaving machine directly enters the coil breaking device 200, the melt-blowing device 300 and the cooling device 400, and the processed mesh 100 is collected by the reeling device, so that the intermediate reeling and unreeling working procedures are reduced, thereby further improving the production efficiency and reducing mesh loss.

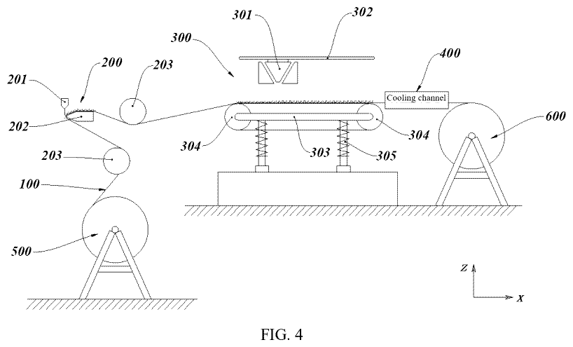

This example further discloses a preparation device for a micro-hook of a suspension belt with a fixing function, wherein the preparation device for the micro-hook of the suspension belt with the fixing function is made by adopting the above preparation method for the micro-hook. As shown in FIG. 4, the preparation device for the micro-hook of the suspension belt with the fixing function of this example includes a coil breaking device 200, a melt-blowing device 300, a cooling device 400, a mesh unreeling device 500 and a mesh reeling device 600, wherein the coil breaking device 200, the melt-blowing device 300 and the cooling device 400 are sequentially arranged between the mesh unreeling device 500 and the mesh reeling device 600, a roll of mesh 100 is mounted on the mesh unreeling device 500, and one end of the mesh 100 is sequentially wound around the coil breaking device 200, the melt-blowing device 300 and the cooling device 400 and then connected to the mesh reeling device 600; one surface of the mesh 100 is provided with coils 101, and the mesh 100 moves under the action of the mesh unreeling device 500 and the mesh reeling device 600; the coil breaking device 200 is used to break the coils 101 of the mesh 100 from middle parts to form two short burrs 102 after each of the coils 101 is broken; the melt-blowing device 300 is provided with a melt-blowing die head 301 controlled to move along X-axis and Y-axis directions, the melt-blowing die head 301 is used to perform melt-blowing on a surface of the mesh 100 with the short burrs 102 to form irregular fluff 103, and the fluff 103 adheres to surfaces of the short burrs 102 and the mesh 100; and the cooling device 400 is used to cool the mesh 100 after being subjected to melt-blowing to solidify the fluff 103, and the made mesh of the suspension belt is wound on the mesh reeling device 600. During working, the mesh unreeling device 500 unreels while the mesh reeling device 600 reels, so that the mesh 100 sequentially moves on the coil breaking device 200, the melt-blowing device 300 and the cooling device 400 at a certain speed. The mesh 100 firstly passes through the coil breaking device 200 to break the coils 101 to form the short burrs 102, then is subjected to surface melt-blowing at the melt-blowing device 300 to form a self-fixing surface with the fluff 103, and then is cooled by the cooling device 400 to solidify the fluff 103 which is firmly combined on the mesh 100 and the short burrs 102.

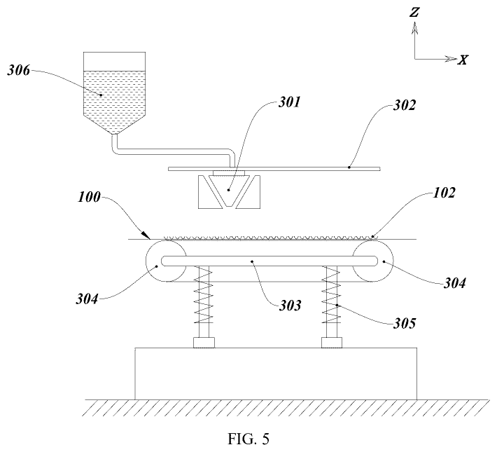

Referring to FIG. 5, in the melt-blowing device 300, the melt-blowing die head 301 is mounted on an XY-axis moving mechanism 302, the XY-axis moving mechanism 302 may drive the melt-blowing die head 301 to move in the X-axis and Y-axis directions, a ball screw drive mechanism may be specifically adopted as the XY-axis moving mechanism 302, and the movement in the X-axis and Y-axis directions may be independently controlled; and a bracket 303 is arranged below the melt-blowing die head 301, a transmission mechanism 304 is arranged on the bracket 303, the transmission mechanism 304 may be a belt conveying mechanism which can drive the mesh 100 to move horizontally, a height adjustment mechanism 305 used to adjust a height of the transmission mechanism 304 is further arranged at a lower part of the bracket 303, and an adjustment screw and other structures may also be adopted as the height adjustment mechanism 305, so that a melt-blowing effect can be ensured by adjusting a melt-blowing distance, and the melt-blowing uniformity can be improved by cooperating with the X-axis and Y-axis reciprocating movement of the melt-blowing die head 301. During melt-blowing, the mesh 100 has a moving speed of 3-10 mm/s on the transmission mechanism 304, and the melt-blowing die head 301 has a reciprocating moving speed of 3-10 mm/s in the X-axis direction, a reciprocating moving speed of 0.5-5 mm/s in the Y-axis direction, and a reciprocating moving amplitude of 3-10 mm in the Y-axis direction, so that full-range melt-blowing can be performed on the mesh by utilizing the reciprocating movement of the melt-blowing die head 301, thereby ensuring the melt-blowing uniformity.

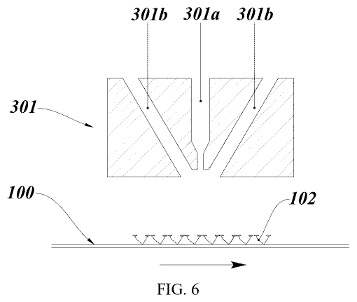

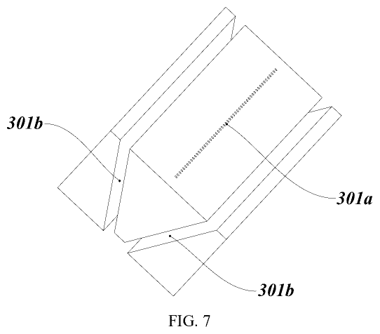

As shown in FIG. 6 and FIG. 7, a plurality of melt channels 301a are uniformly distributed on the melt-blowing die head 301 along the Y-axis direction, the melt channels 301a are connected to melt material barrels 306, the melt-blowing die head 301 is further provided with hot air channels 301b positioned in two sides of the melt channels 301a, and the hot air channels 301b are connected to hot wind delivery devices. The melt material barrel 306 can melt a melt-blowing material into a fluid state. A melt material is sucked by adopting a negative-pressure suction force generated by high-pressure hot air passing through the melt-blowing die head 301, and the high-pressure hot air purges the melt material to enable the melt material to be diverged and sprayed outwards, thereby being conducive to improving the melt-blowing uniformity and the irregularity of the formed fluff; meanwhile, the high-pressure hot air can maintain a temperature of the melt-blowing die head 301, thereby ensuring the fluidity of the melt material and preventing the blockage of the melt channels. The hot air channels 301b in the two sides are formed obliquely, which can enable the melt-blowing die head 301 to spray in a fan shape, thereby increasing the melt-blowing area and uniformity.

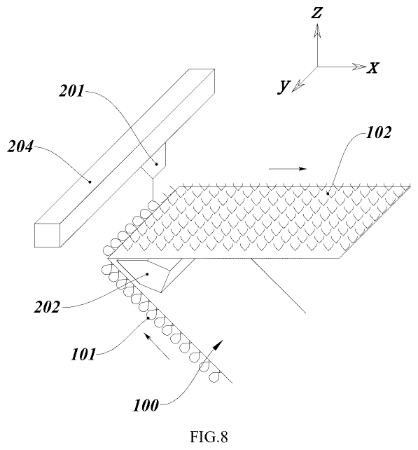

As shown in FIG. 4 and FIG. 8, in this example, the coil breaking device 200 is preferably a laser cutting machine, including a laser cutting head 201, a supporting plate 202, a guide roller 203 and a Y-axis moving mechanism 204, the mesh 100 is tensioned on the supporting plate 202 arranged along the Y-axis through the guide roller 203, and a contact end of the supporting plate 202 with the mesh 100 is of a sharp-angle structure, so that the mesh 100 positioned at a sharp-angle position of the supporting plate 202 is in a “<” shape, so as to separate the coils 101 at the sharp-angle position from the coils 101 at other positions, and maintain the coils 101 to be unfolded towards a cutting direction; the laser cutting head 201 is mounted on the Y-axis moving mechanism 204, the ball screw drive mechanism may also be adopted as the Y-axis moving mechanism 204, and the laser cutting head 201 is driven by the Y-axis moving mechanism 204 to move along the sharp-angle position of the supporting plate 202 to cut off the coils 101 on the mesh 100; and during laser cutting of the coils 101, the mesh 100 has a moving speed of 3-10 mm/s on the supporting plate 202, an input power of the laser cutting head 201 is controlled at 10-30 W, a Y-axis reciprocating moving speed is controlled at 10-30 mm/s, and a Y-axis cutting stroke of the laser cutting head 201 is 250-450 mm. By adopting the above laser cutting machine, a cutting speed is higher, coil breaking is more complete and stable, and a power of laser cutting may be reduced by cooperating with the melt-blowing process, thereby being conducive to reducing the risk of cutting a mesh substrate and ensuring the stability of the processing quality of the mesh of the suspension belt.

In this example, the mesh 100 after being subjected to melt-blowing may be cooled at a temperature controlled at 0-25° C. in a natural cooling manner or by adopting the cooling device 400. During cooling by adopting the cooling device 400, the cooling device 400 may be provided with a cooling channel for the mesh 100 to pass through, the cooling channel is internally provided with a plurality of air inlets connected to air blasting equipment, and the mesh 100 after being subjected to melt-blowing is rapidly cooled in a forced air cooling manner, so as to facilitate the rapid shaping of the fluff 103.

For the preparation method and the preparation device for the micro-hook of the suspension belt with the fixing function of the present disclosure, by organically combining the coil breaking and melt-blowing processes, the irregular fluff is formed on the surfaces of the short burrs formed by coil breaking and the mesh, thereby increasing the frictional force of the mesh, improving the gripping force of the suspension belt made of the mesh in the body, reducing the risk of the mesh of the suspension belt sliding on the tissue, reducing the suture fixation of the suspension belt during the operation, and saving the operation time; in addition, due to the existence of the melt-blown fluff, the requirements for the shapes and sizes of the barbs on the short burrs are reduced, and the efficiency and process flexibility of the coil breaking working procedure can be improved, thereby being conducive to improving the overall production efficiency of the mesh and the suspension belt.

In addition, the mesh made by the above preparation method and preparation device for the micro-hook not only may be used to make a suspension belt for urinary incontinence, but also may be used to make a medical repair patch such as a hernia repair patch.

The present disclosure and the embodiments thereof have been schematically described above, the description is not restrictive, what is shown in the accompanying drawings is only one of the embodiments of the present disclosure, and the actual structure is not limited thereto. Therefore, if those ordinarily skilled in the art are inspired thereby, and without departing from the creative purpose of the present disclosure, design structural modes and examples similar to the technical solution without creative work, all such structural modes and examples shall fall within the protection scope of the present disclosure.

1. A preparation device for a micro-hook of a suspension belt with a fixing function, comprising a coil breaking device, a melt-blowing device, a cooling device, a mesh unreeling device and a mesh reeling device,
wherein the coil breaking device, the melt-blowing device and the cooling device are sequentially arranged between the mesh unreeling device and the mesh reeling device, a roll of mesh is mounted on the mesh unreeling device, and one end of the mesh is sequentially wound around the coil breaking device, the melt-blowing device and the cooling device and then connected to the mesh reeling device; one surface of the mesh is provided with coils, and the mesh moves under an action of the mesh unreeling device and the mesh reeling device; the coil breaking device is used to break the coils of the mesh from middle parts to form two short burrs after each of the coils is broken; the melt-blowing device is provided with a melt-blowing die head controlled to move along X-axis and Y-axis directions, the melt-blowing die head is used to perform melt-blowing on a surface of the mesh with the short burrs to form irregular fluff, and the fluff adheres to surfaces of the short burrs and the mesh; and the cooling device is used to cool the mesh after being subjected to melt-blowing to solidify the fluff;
wherein, in the melt-blowing device, the melt-blowing die head is mounted on an XY-axis moving mechanism, a bracket is arranged below the melt-blowing die head, a transmission mechanism is arranged on the bracket, and a height adjustment mechanism used to adjust a height of the transmission mechanism is further arranged at a lower part of the bracket; and during melt-blowing, the mesh has a moving speed of 3-10 mm/s on the transmission mechanism, and the melt-blowing die head has a reciprocating moving speed of 3-10 mm/s in the X-axis direction, a reciprocating moving speed of 0.5-5 mm/s in the Y-axis direction, and a reciprocating moving amplitude of 3-10 mm in the Y-axis direction; and
wherein a plurality of melt channels are uniformly distributed on the melt-blowing die head along the Y-axis direction, the melt channels are connected to melt material barrels, the melt-blowing die head is further provided with hot air channels positioned in two sides of the melt channels, and the hot air channels are connected to hot wind delivery devices.

2. The preparation device according to claim 1, wherein the coil breaking device is a laser cutting machine, comprising a laser cutting head, a supporting plate, a guide roller and a Y-axis moving mechanism, the mesh is tensioned on the supporting plate arranged along the Y-axis through the guide roller, a contact end of the supporting plate with the mesh is of a sharp-angle structure, so that the mesh positioned at a sharp-angle position of the supporting plate is in a “<” shape, the laser cutting head is mounted on the Y-axis moving mechanism, and the laser cutting head is driven by the Y-axis moving mechanism to move along the sharp-angle position of the supporting plate to cut off the coils on the mesh; and during laser cutting of the coils, the mesh has a moving speed of 3-10 mm/s on the supporting plate, an input power of the laser cutting head is controlled at 10-30 W, a Y-axis reciprocating moving speed is controlled at 10-30 mm/s, and a Y-axis cutting stroke of the laser cutting head is 250-450 mm.

3. A preparation method for a micro-hook of a suspension belt with a fixing function, using the preparation device of claim 1, comprising the steps of:
coil breaking wherein one surface of a mesh being provided with coils, and breaking the coils of the mesh from middle parts by utilizing a coil breaking device to form two short burrs after each of the coils is broken;
surface melt-blowing wherein performing melt-blowing on a surface of the mesh with the short burrs by utilizing a melt-blowing device to form irregular fluff, and the fluff adhering to surfaces of the short burrs and the mesh; and
cooling wherein cooling the mesh after being subjected to melt-blowing by utilizing a cooling device or in a natural cooling manner to solidify the fluff.

4. The preparation method according to claim 3, wherein the coil breaking device is a laser cutting machine, a high-temperature melting and ironing machine, a cutting machine or a grinding machine.

5. The preparation method according to claim 3, wherein a melt-blowing material is the same as a material of the mesh.

6. The preparation method according to claim 3, wherein the coil breaking device in step S1 melts and breaks the coils through a high temperature, and melts ends of the short burrs to form barbs.

7. The preparation method according to claim 3, wherein the mesh sequentially passes through the coil breaking device, the melt-blowing device and the cooling device, and the mesh moves continuously or intermittently among the coil breaking device, the melt-blowing device and the cooling device to sequentially complete coil breaking, surface melt-blowing and cooling working procedures.

8. The preparation method according to claim 7, wherein the mesh is woven by a weaving machine, the mesh woven by the weaving machine directly enters the coil breaking device, the melt-blowing device and the cooling device, and the mesh is collected by a mesh reeling device.
